(19) United States
(12) Patent Application Publication
(10) Pub. No.: US 2009/0303430 A1
(43) Pub. Date: Dec. 10, 2009

(54) WIND POWERED SPORT GOGGLES

(76) Inventor: JOSEPH ALLEN GREGORY, BOISE, ID (US)

Correspondence Address: DYKAS, SHAVER & NIPPER, LLP, P.O. BOX 877, BOISE, ID, 83701-0877, US

(21) Appl. No.: 12/135,409

(22) Filed: Jun. 9, 2008

(51) Int. Cl. A61F 9/02

(52) U.S. Cl. 351/62

(57) ABSTRACT

Sport goggles are disclosed which includes air scoops and air exhausting tubes for directing air through the goggle body. The device may include a venturi powered air vent to remove air from within the goggles, and may include a fan turned by airflow, for powering electrical devices associated with the goggles.

### FIELD OF THE INVENTION

This invention generally relates to sport goggles, and more particularly to sport goggles with an anti-fogging mechanism caused by air flow.

### BACKGROUND OF THE INVENTION

Goggles are used in a number of sports, and are considered essential for sports such as skiing, snowboarding, motorcycle riding, race car driving, and many other active sports. Goggles for sports such as these typically include a lens, which can be one or two layers of plastic separated by an air space. These lenses are held by the goggle body, which holds the double lens in its spaced apart relationship. In the case of winter sport goggles, the purpose of the double lens and the air space is to allow the inner lens to be warmer, so as to reduce the formation of fog on the inner lens. Sport goggles such as these enclose a region of air around the user's eyes. Since this air is warmer and moister than the surrounding air, if the inner lens of the goggle is cold or cool, fog formation may occur.

Reduction or removal of fog is the subject of a number of different goggle designs. One way to reduce fog is by the double layer lens approach described above. Other goggles use a more active strategy, with electric powered fans being provided on the inside of the goggles to blow air around the inside of the goggles and reduce fogging.

Another strategy in fog prevention and removal is to provide air vents around the frame of the goggles so that as the user moves through the air, there is a circulation of air through the goggles. The purpose of the air circulation is to remove more moisture from inside the goggles, and thus to reduce fog.

None of these strategies fully solves the problem of preventing fog formation on the inside of goggles. The current invention addresses the problem of fog formation on the inside of sport goggles.

### SUMMARY OF THE INVENTION

These and other goals are accomplished by the sport goggles of the invention. When sport goggles of the invention are discussed, it is in reference to a singular item: the wind powered sport goggles. The sport goggles of the invention includes a goggle body, a lens, one or more air scoops built into the goggle body, one or more air vent openings, with each air vent opening associated with an air exhausting tube. The goggle body defines an opening in which the lens is placed. The lens has a left and right side edge, a lens top edge, and a lens bottom edge. Portions of the goggle surrounding the lens may form ridges for directing air flow towards the sides of the lens and into the air scoops. The goggles are designed to be placed over a user's face, so that the eyes are protected from wind and blowing snow. In position on a user's head, the goggles form an enclosed air space over the user's eyes.

The goggle body would typically have an attached head strap, which would serve to hold the goggles on to the user's head. The head strap could also be configured to attach to a helmet, such as a ski helmet, or to wrap around the back of the helmet. The goggle body defines a lens holding groove, into which the lens of the goggle is placed and is secured. The goggle body also has one or more air scoops. Associated with each air scoop is an air exhausting tube. When the user is in motion, air strikes the front of the lens and is directed toward the left and right side of the goggles. The air scoops would typically be positioned adjacent to the left and right edges of the lens, and would admit the air directed towards the air scoop. The flow of air directed at the air scoops would be routed through the air exhausting tube, and after flowing a short distance through the goggle body would be exhausted at another point in the goggle body, such as the rear, top or bottom of the goggle body.

Associated with each air exhausting tube is one or more air vent openings, with each air vent opening in communication with the enclosed air space between the lens and the user's face. As air is directed through the goggle body in the air exhausting tubes, the air rushes over the air vent openings, and causes air to be drawn through the air vent openings from the enclosed air space. The flow of air through the air vent openings is due to a venturi effect as the air rushes through the air exhausting tube. As air velocity increases due to a constriction in the air exhausting tube, air pressure in the air vent decreases and air is thus withdrawn from the enclosed air space of the goggles.

The sport goggle body would also have one or more air intakes, such as on the edge of the sport goggle body below the bottom edge of the lens. As the user moves through the air, air enters an air intake, flows across the inside surface of the lens, and exits through an air vent opening on the inside of the goggle, and from there is removed from the goggle body.

An optional feature of the sport goggles of the invention is to include one or more rotatable fans or turbines within the sport goggle body. The fans would be configured to be rotated by movement of air through the air exhausting tube or tubes. Preferably, one rotatable fan would be positioned on the left side and one on the right side of the sport goggle body. These fans could be configured with an axis of rotation which is either parallel with the long axis of the lens, or parallel with the short axis of the lens. The long axis of the lens would generally extend from the left to the right side of the lens edge, and the short axis of the lens would extend from the top to the bottom of the lens.

An additional feature of the sport goggles of the invention can be one or more small generators of electricity which are associated with one or more fans, as described above. The generators would be turned by the rotation of the fans, and would create an electric current by the rotation of the fan blades. The electric current produced by the generators would be utilized to power electrical devices associated with the sport goggles. This could be through the use of a battery, which would be charged by the electric current. The energy produced by the generators could be utilized to power a heating device associated with the lens, such as a conductive strip for removing fog from the lens by heating the lens. Other electrical devices could be powered such as music playing devices, radios, or wireless communication. A USB port could be included in the goggle for charging devices, such as music playing devices, when electricity is available.

These features of the sport goggles of the invention could include a goggle which is built integrally with a helmet, in which the helmet would form the goggle body, with the structures described above being located within the shell of the helmet.

The purpose of the foregoing Abstract is to enable the public, and especially the scientists, engineers, and practitioners in the art who are not familiar with patent or legal terms or phraseology, to determine quickly from a cursory inspection, the nature and essence of the technical disclosure of the application. The Abstract is neither intended to define the invention of the application, which is measured by the claims, nor is it intended to be limiting as to the scope of the invention in any way.

Still other features and advantages of the present invention will become readily apparent to those skilled in this art from the following detailed description describing preferred embodiments of the invention, simply by way of illustration of the best mode contemplated by carrying out my invention. As will be realized, the invention is capable of modification in various obvious respects all without departing from the invention. Accordingly, the drawings and description of the preferred embodiments are to be regarded as illustrative in nature, and not as restrictive in nature.

### BRIEF DESCRIPTION OF THE DRAWINGS

FIG. 1 is a front view of the sport goggles of the invention.

FIG. 2 is a cross-sectional top view of the sport goggles of the invention.

FIG. 3 is a front view of the sport goggles of the invention showing the channeling of air into an air scoop.

FIG. 4 is a perspective view of the anti-fogging system of the sport goggles of the invention.

FIG. 5 is a perspective view of a generator and fan (vertical axis).

FIG. 6 is a perspective view of a generator and fan (horizontal axis).

FIG. 7 is a cross-sectional top view of the sport goggles of the invention when built into a helmet.

### DESCRIPTION OF THE PREFERRED EMBODIMENTS

While the invention is susceptible to various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

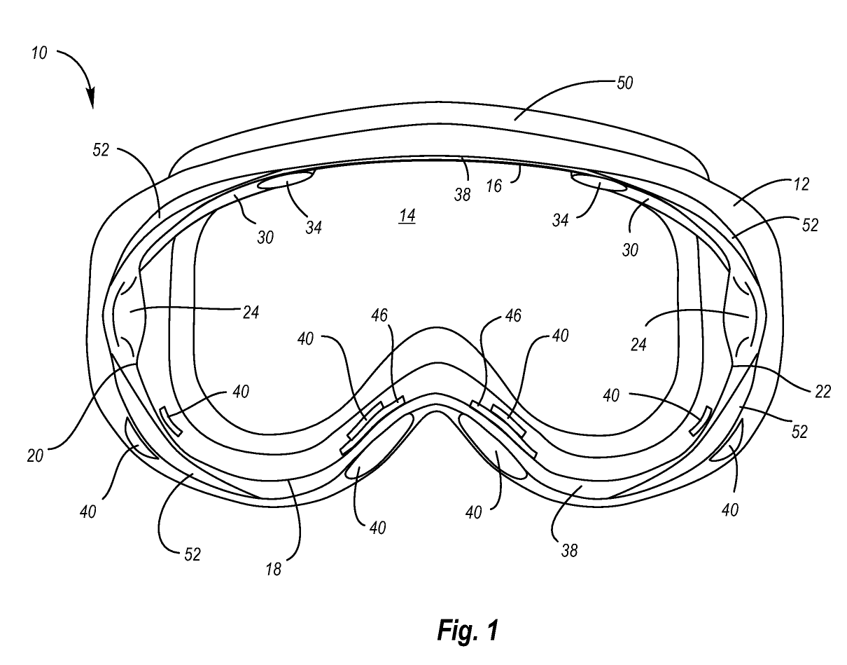

One or more of the preferred embodiments of the sport goggles of the invention are shown in FIGS. 1-7.

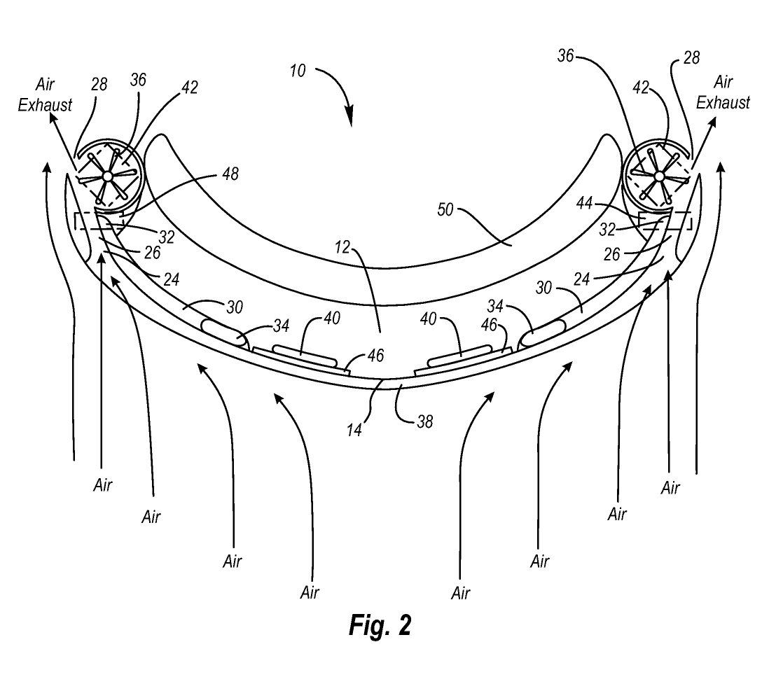

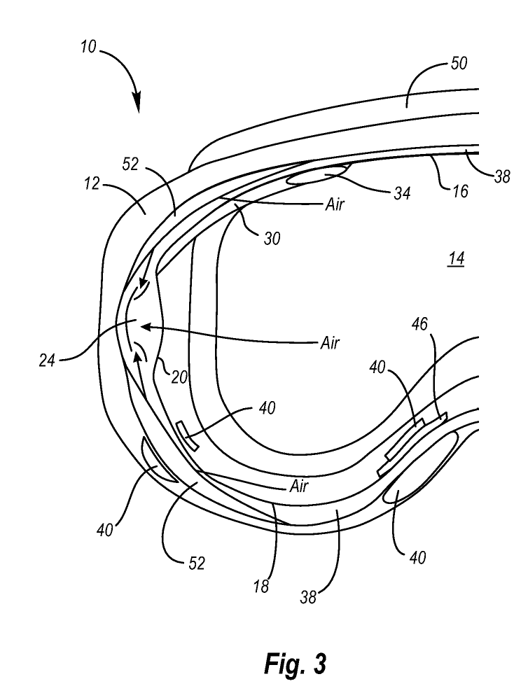

FIG. 1 shows the goggle body 12 which surrounds the lens 14. The lens 14 is held in place in a lens groove formed in the goggle body 12. The lens 14 includes a lens top edge 16, a lens bottom edge 18, a lens right side 20, and a lens left side 22. Adjacent to the lens right side 20 and the lens left side 22 are air scoops 24. As the user of the goggles moves through the air, air strikes the front of the lens 14 as shown in FIGS. 2 and 3, and air is directed towards the left and right side of the goggles. The air scoops are directly in the path of the redirected air flow, and air is thus forced into the air scoops 24. Air redirects 52 are shaped ridges which help to direct air into the air scoops 24.

FIG. 1 shows an air vent 30, which is a tube on the inside of the goggle body 12, which has an opening at either of its ends. In FIG. 1, the air vent opening to the inside of the goggle is visible and is numbered 34. At the opposite end of the air vent is an opening to the air exhausting tube, which is numbered 32, as shown in FIG. 2. FIG. 1 shows a ridge 38, which is a raised ridge along the top and bottom of the goggle body adjacent to the lens 14. The ridge 38 serves to channel air toward the left and right side of the lens and into an air scoop 24. Also shown in FIG. 1 are several air intakes 40 which are passages through the goggle body 12 into the interior of the goggle body. The air intakes 40 serve to provide a flow of outside air into the goggle body. The flow of outside air into the goggle body is facilitated by movement through the air by the user, and also by removal of air from inside the goggle body by the air vent 30. Also shown in FIG. 1 are two examples of heating elements 46. The heating elements 46 would be placed inside the goggle and adjacent the lens in order to provide heat to facilitate defogging of the lens 14. FIG. 1 also shows a region of face foam 50, which is attached to the goggle body 12, and which is made to fit against the user's face in order to form an airtight seal. Also shown in FIG. 1 is an air re-direct 52, which is a curving of the ridge 38, and serves to more effectively direct air towards the air scoops 24. Air re-directs 52 are positioned in each of the four corners of the goggle, on either side of the air scoops 24.

FIG. 2 is a cross-sectional top view showing of the interior mechanisms of an air scoop 24. As air enters the air scoop 24, it is directed into an air exhausting tube 26. Since air is collected in the air scoop from a wide area and funneled into the smaller diameter air exhausting tube 26, the speed of the air increases. The air continues through the air exhausting tube 26 and exits via the exhaust opening 28 in the goggle body. The air exhausting tube 26 can be angled to direct air around a helmet as it exits the air exhaust opening 28. Located along the air exhausting tube 26 is an opening to the air vent 30. The air vent 30 is a short tube which has an opening 32 in the air exhausting tube 26 and an opening 34 in the enclosed space inside the sport goggles 10. Since air is moving rapidly in the air exhausting tube 26, as it passes over the opening 32 of the air vent 30, it draws air from the enclosed space into the stream of air in the air exhausting tube 26. The air vent 30 is a tube routed to a location inside the goggles for optimum removal of moisture and air from inside the sport goggles 10.

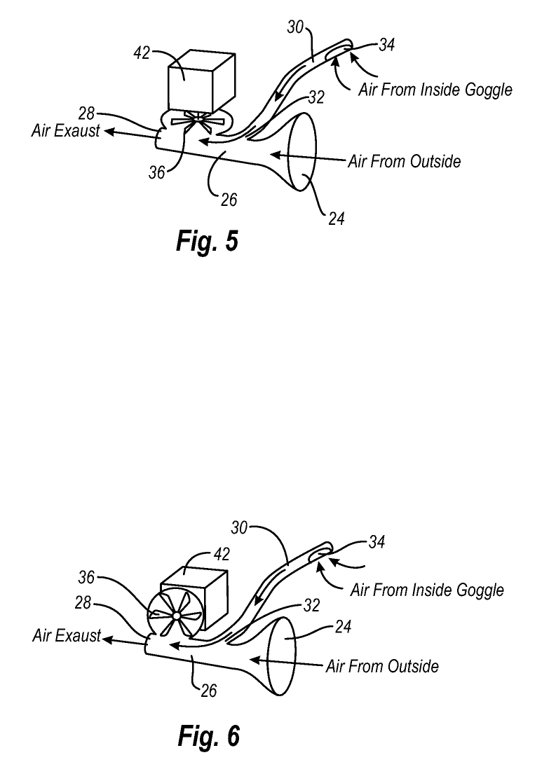

FIG. 2 shows one preferred configuration of the fans 36 which turn as air passes through the air exhausting tube 26. Air enters from the air scoop 24 and turns the fan 36 as it passes through the goggle body 12. In the configuration of FIG. 5, the axis of rotation of the fans 36 is parallel with the short axis of the lens 14. In the configuration of FIG. 6, the axis of rotation of the fans 36 is parallel with the long axis of the lens 14. Accessories associated with the fan may include a generator, which is shown as 42 in FIGS. 2, 5, and 6. The generator 42 in conjunction with the battery 44 may be used for storage of electrical energy. Electrical devices can be powered by the generator 42 or by the battery 44. An example of such an electrical device is the heating element 46 which is shown in FIGS. 1, 2, 3, and 4. Such a heating element would produce heat adjacent to or within the lens 14, and thus dissipate fog. Also shown in FIG. 2 is a USB port 48. The USB port 48 can accept electrical energy from the battery 44 or directly from the generators 42 for powering USB-supported devices such as music playing devices.

FIG. 3 shows a front view of one side of the sport goggles of the invention. In this view, more detail is show of the air re-direct 52 which is part of the ridge 38. Shown in FIG. 3 are routes of air which are channeled by the ridges 38 and the air re-directs 52 towards the air scoop 24.

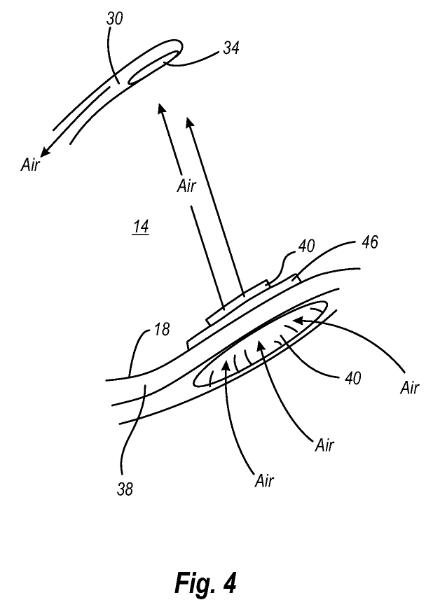

FIG. 4 shows the defogging action of the invention. As the user moves through the air, air enters an air intake 40, flows across the inside surface of the lens 14, and exits through an air vent 30 from the goggle body 12. Air is drawn through the air vent opening 34 by the venturi effect created by air movement through the air exhausting tube 26. Shown also is a heating element 46 which can aid in defogging.

FIG. 5 is a view showing more detail of the air channeling mechanisms of the device. Shown in FIG. 5 is the air scoop 24, which is exposed on the front face of the goggles. Air is channeled into the air scoop 24, proceeds through the air exhausting tube 26, and out the air exhaust opening 28. As it passes through this route, it passes by the air vent opening to the air exhausting tube, labeled as 32, and by the action of rapidly flowing air moving over the air vent opening 32, air is drawn into the opening inside the goggle 34, and through the air vent tube 30. This serves to remove air from inside the goggles and direct it through the air exhaust opening 28 to the outside of the goggles. The fan 36 and the generator 42 are shown in FIG. 5. In this figure, the axis of the fan is in a vertical orientation which is perpendicular to the long axis of the goggles.

FIG. 6 shows a similar structure as FIG. 5 but shows the fan 36 and generator 42 in an orientation in which the axis of the fan is parallel to the long axis of the goggles.

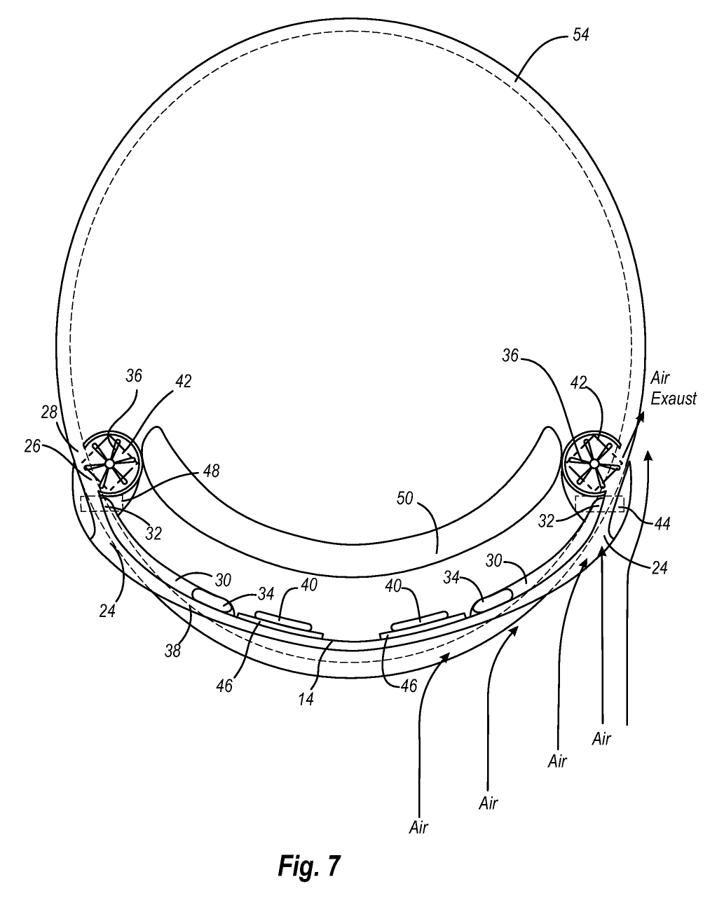

FIG. 7 shows a version of the sport goggles of the invention in which the goggle body 12 is incorporated into a helmet 54. The internal structures of the goggle are the same as those shown in FIG. 2.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

What is claimed is:

1. A sport goggle with a lens, said lens having lens side edges, a lens top edge, and a lens bottom edge, for directed airflow, with said goggles configured to form an enclosed air space over a user's eyes, said sport goggles comprising:
a goggle body for holding said lens;
one or more air scoops positioned in said goggle body adjacent to an edge of said lens, with said one or more air scoops connected to an air exhausting tube;
one or more air vent openings in said air exhausting tube, with said air vent in communication with said enclosed air space; wherein
said goggle body is configured to direct air into said one or more air scoops when air strikes the front surface of said lens, with said air exhausting tube configured to channel air through said goggle body, with said air exhausting tube configured to direct air across said one or more air vent openings, for removal of air from said enclosed space by a venturi effect.

2. The sport goggles of claim 1 which further comprises an air scoop on each side of said goggle body, each with an air exhausting tube and an air vent.

3. The sport goggles of claim 1 which further comprises a defogging capability due to removal of air from said enclosed space by said one or more air vents.

4. The sport goggles of claim 1 which further comprises air intake vents in said goggle body.

5. The sport goggles of claim 4 in which said air intake and said air vent are configured to work together to enhance airflow to move air across said lens, and to remove air and moisture from said lens surface.

6. The sport goggles of claim 4 in which said air intake vents are positioned in said goggle body adjacent to said lens bottom edge.

7. The sport goggles of claim 1 in which said air exhausting tube narrows from said air scoop toward an air exhaust opening, for increasing the velocity of said air in said air exhausting tube.

8. A sport goggle with a lens, said lens having lens side edges, a lens top edge, and a lens bottom edge, for directed airflow, with said goggle configured to form an enclosed air space over a user's eyes, said sport goggles comprising:
a goggle body for holding said lens;
a pair of air scoops positioned in said goggle body adjacent to the sides of said lens, with each of said air scoops connected to an air exhausting tube in which said air exhausting tube narrows from said air scoop toward an air exhaust opening, for increasing the velocity of said air in said air exhausting tube;
one or more air intakes defined in said goggle body, for admitting air into said enclosed air space;
at least one air vent opening in each of said air exhausting tubes, with said air vents in communication with said enclosed air space; wherein
said goggle body is configured to direct air into said one or more air scoops when air strikes the front surface of said lens, and said air exhausting tubes configured to direct said air through said goggle body, with said airflow through said air exhausting tubes being directed across said air vent openings, for removal of air from said enclosed space by a venturi effect, with said air intakes configured to admit replacement air into said enclosed air space.

9. The sport goggles of claim 8 in which said air intake and said air vent are configured to work together to enhance airflow to move air across said lens, and to remove air and moisture from said lens surface.

10. The sport goggles of claim 8 which further comprises one or more rotatable fans within said sport goggle body, configured for rotation by air movement through said air exhausting tube.

11. The sport goggles of claim 8 which further comprises a rotatable fan on either side of said sport goggle body.

12. The sport goggles of claim 10 in which said one or more rotatable fans is oriented with an axis of rotation generally parallel with a long axis of said lens.

13. The sport goggles of claim 10 in which said one or more rotatable fans is oriented with an axis of rotation generally parallel with a short axis of said lens.

14. The sport goggles of claim 10 which further comprises one or more generators associated with said one or more fans, with said generators configured to produce an electric current when said one or more fans are turned by air movement in said air exhausting tube.

15. A sport goggle with a lens, said lens having side edges, a lens top edge, and a lens bottom edge, for directed airflow, with said goggle configured to form an enclosed air space over a user's eyes, said sport goggles comprising:
a goggle body for holding said lens;
one or more air scoops positioned in said goggle body adjacent to an edge of said lens, with said one or more air scoops for directing air into one or more air exhausting tubes;
one or more rotatable fans positioned adjacent a side of said sport goggle body, configured for rotation by air movement through said air exhausting tube; and
one or more air vent openings in said air exhausting tube, with said air vent in communication with said enclosed air space; wherein
said goggle body is configured to direct air into said one or more air scoops when air strikes the front surface of said lens, with said air exhausting tube configured to direct air through said goggle body, with said air exhausting tube configured to direct air across said one or more air vent openings, for removal of air from said enclosed space by a venturi effect.

16. The sport goggles of claim 15 which further comprises a rotatable fan on each side of said sport goggle body.

17. The sport goggles of claim 15 in which said one or more rotatable fans is oriented with an axis of rotation generally parallel with a long axis of said lens.

18. The sport goggles of claim 15 in which said one or more rotatable fans is oriented with an axis of rotation generally parallel with a short axis of said lens.

19. The sport goggles of claim 15 which further comprises one or more generators associated with said one or more fans, with said generators configured to produce an electric current when said one or more fans are turned by air movement in said air exhausting tube.

20. The sport goggles of claim 19 which further includes an electrical device powered by said one or more generators.

21. The sport goggles of claim 20 in which said electrical device is a heating device associated with said lens for removing fog from said lens.

22. The sport goggles of claim 19 which further includes a USB port for attachment of an electrical device, such as a music playing device, for charging said device.

23. The sport goggles of claim 19 which further includes one or more batteries for storage of electricity generated by said generators.

24. A sport goggle with a lens, said lens having side edges, a lens top edge, and a lens bottom edge, with said goggle configured to form an enclosed air space over a user's eyes, said sport goggles comprising:
a goggle body for holding said lens;
one or more air scoops positioned in said goggle body adjacent to an edge of said lens, with said one or more air scoops connected to one or more air exhausting tubes;
one or more rotatable fans positioned adjacent a side of said sport goggle body, configured for rotation by air movement through said one or more air exhausting tubes;
one or more air vent openings in said air exhausting tube, with said air vent openings in communication with said enclosed air space;
one or more air intakes, for admitting air into said enclosed air space, with said air vents and said air intakes configured to work together to enhance airflow to move air across said lens, and to remove air and moisture from said lens surface; and
one or more generators associated with said one or more fans, with said generators configured to produce an electric current when said one or more fans are turned by air movement in said air exhausting tube; wherein
said goggle body is configured to direct air into said one or more air scoops when air strikes the front surface of said lens, with said air exhausting tube configured to channel air through said goggle body, with said air exhausting tube configured to direct air across said one or more air vent openings, for removal of air from said enclosed space by a venturi effect, with said air intakes directing air across an inside surface of said lens, with said air from said air scoops for moving air to turn said fans and said generators for generation of electricity.
